(19) United States
(12) Patent Application Publication
(10) Pub. No.: US 2002/0197240 A1
(43) Pub. Date: Dec. 26, 2002

(54) Marrow stem cell (MSC) transplantation for use in tissue and/or organ repair

(76) Inventor: Ray C. Chiu, Montreal (CA)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/146,092

(22) Filed: May 15, 2002

Related U.S. Application Data
(60) Provisional application No. 60/290,670, filed on May 15, 2001.

(51) Int. Cl. A61K 45/00; C12N 5/08

(52) U.S. Cl. 424/93.7; 435/372

(57) ABSTRACT

The present invention relates to a method of inducing tissue cell growth and/or organ repair in vivo without eliciting an immune response. The method includes the transplantation of undifferentiated stem cells into a recipient suffering from tissue and/or organ damage. The source of the undifferentiated stem cells for the transplantation may be autologous, allogenic or xenogenic. Preferably, the undifferentiated stems cells are marrow stroma cells.

### BACKGROUND OF THE INVENTION

(a) Field of the Invention

The invention relates to undifferentiated stem cells and the use of these cells in improving tissue cell growth and/or organ repair. A preferred aspect of the invention relates to allo-/xeno-transplantation of bone marrow stroma cells (MSCs) to elicit tissue and/or organ repair in vivo. The present invention also relates, in accordance with one aspect, to the transplantation of MSCs into the myocardium to grow new muscle fibers.

(b) Description of Prior Art

In spite of tremendous improvement in medical technology and therapy, critically ill patients such as those who suffer from severe trauma and sepsis continue to die in intensive care units of hospitals throughout the world. The common pathway of how these patients die is known as ‘multiple organ failure’, in which many vital organs in the body, such as the kidney, lung, liver, heart and brain fail in their function one after another. Such failures are caused by or will cause tissue damage of these organs, with loss of functioning cells by necrosis or apoptosis. When sufficient organ damage occurs, the organ loses the reserve to recover, even with the application of an organ support system as available today in our intensive care units (such as ventilator and dialysis, etc.). Sometimes organ transplantation is indicated, but due to the lack of donors and the need for immunosuppression, in reality this is rarely done. One of the reasons for this is that because of the scarcity of donor organs, there is a reluctance to select a critically ill patient with multiple organ failures as a recipient, since the demise of such a patient would mean the loss of a precious donor organ. In recent years, studies by us and others have shown that bone marrow stromal cells which can be obtained by bone marrow aspiration, a routine clinical procedure, and culture expanded in vitro to multiply the cell population, can be used to regenerate damaged organs by injecting them to specific sites. Our recent study indicates that these cells can undergo site specific differentiation, developing into mature functioning cells guided by their micro-environment (Wang et al. Journal of Thoracic and Cardiovascular Surgery. Vol. 120, No. 5, Nov. 2000).

In recent years, tissue engineering has been suggested as another exciting approach to treating organ failure and damage, in which various cells are cultured in vitro over biodegradable polymer scaffolds to create a 3-dimensional construct in vitro, which can then be implanted to replace damaged tissues or organs. Upon the absorption of a biodegradable scaffold in vivo, these replacement tissues would not require immunosuppression if autologous donor cells were used for tissue engineering. Advances are being made in constructing cardiovascular structures, such as arteries and cardiac valves. Attempts are also being made to engineer 3-dimensional myocardial tissue blocks by seeding cardiac myocytes on a 3-dimensional scaffold, and cultured in rotating bioreactors. Without concomitant creation of a coronary vascular system within these constructs, however, such tissue-engineered myocardium cannot be used therapeutically in vivo, as they will suffer rapid ischemic necrosis.

Such difficulties described above may be circumvented by tissue engineering neomyocardium in vivo. As is well known, cardiomyocyte loss from myocyte necrosis and apoptosis plays an important role in the initiation and progression of heart failure (Olivetti G, Abbi R, Quaini F, et al.: Apoptosis in the failing human heart. N Engl J Med 1997;336:1131-1141).

Cellular cardiomyoplasty is a potential future therapy for heart failure in which donor cells with the potential to differentiate into cardiac myocytes are implanted into the damaged myocardium in order to regenerate new muscle fibers (Chiu R C-J, Zibaitis A, Kao R L: Cellular cardiomyoplasty: Myocardial regeneration with satellite cell implantation. Ann Thorac Surg 1995; 60:12-18). To date, a number of donor cells have been studied by various investigators, and are summarized below.

A. Fetal Cardiomyocytes

Differentiated fetal cardiomyocytes retain a capacity for proliferation. Both in rodent and in canine models, fetal cardiomyocytes implanted into the myocardial wall of adult animals have been shown to be successfully engrafted, and develop into cells which are morphologically and functionally indistinguishable with the native cardiac myocytes within the recipient heart. They form gap junctions which should allow them to be depolarized and contract synchronously as a syncytium (Soonpaa M H, Koh G Y, Klug M G, et al.: Formation of nascent intercalated disks between grafted fetal cardiomyocytes and host myocardium. Science 1994; 264: 98-101). By using fetal cells, however, the problem of donor cell availability becomes more formidable and the ethical issues more complex. Unlike for the use of fetal cells for treatment of neurological diseases, such as Parkinson's disease, in which engraftment of small numbers of cells may be adequate for therapeutic effect, fetal cardiomyocyte transplantation is likely to require millions of new cells to be efficacious, and continued proliferation of engrafted myocytes cannot be expected to expand the population, once they are removed from the donor embryos.

B. Embryonic Stem Cells

Klug et al. transfected a transgene that confers resistance to a toxic drug into embryonic stem cells under the control of a cardiac specific promoter (Klug M. G., Soonpaa M. H., Koh G. Y., Field L. J. Genetically selected cardiomyocytes from differentiating embryonic stem cells form stable intracardiac grafts. J. Clin. Invest. 1996; 98:216-24). When embryoid bodies derived from these stably transfected embryonic stem cells were exposed to the toxic antibodies, only cardiac myocytes survived. These cells were harvested and injected into the myocardial wall of the adult mice, where they engrafted and formed appropriate cell-to-cell junctions, i.e. intercalated discs with desmosomes and gap junctions, with host cardiomyocytes while maintaining a morphologically differentiated state. In principle, this strategy would allow for generating large numbers of donor myocytes. However, the ethical issues of using embryonic stem cells are currently hotly debated.

C. Modified Adult Cardiomyocytes and Myoblast Cell Lines

The adult cardiomyocytes are generally believed to be terminally differentiated and thus unable to proliferate. It has been previously reported that adult cardiomyocytes obtained from biopsy can be induced to proliferate in vitro, while retaining some phenotypic characteristics of the cardiac myocytes, and they can be successfully engrafted into the myocardium (Li R K, Jia Z Q, Weisel R D et al. Cardiomyocyte transplantation improves heart function. Ann. Thor. Surg. 1996; 62:654-660). It was shown that such cells implanted into an ischemic myocardium could improve ventricular function. These findings, however, require independent confirmation, more precise identification of cellular phenotype, and assurance against oncogenicity of the transformed cardiac myocytes before they can be considered for clinical use.

Robinson et al. and other investigators implanted cells from established cell lines, such as C2 C12 cells which were originally derived from skeletal myoblasts (satellite cells) (Robinson S W, Cho P W, Levitsky H I et al.: Arterial delivery of genetically labeled skeletal myobalsts to the murine heart: Long-term survival and phenotypic modification of implanted myoblasts. Cell Transplantation 5:77-91, 1996). There is evidence that such cells, in spite of their origin from skeletal muscle, may transdifferentiate in the heart acquiring certain phenotypic characteristics of a cardiac myocyte, such as the expression of Connexin-43 and the formation of desmosomes at cell junctions. Although it is very convenient to use established cell lines which can be purchased from suppliers, the concern of oncogenicity upon transplantation in vivo, and the need for immunosuppression may limit their application in clinical therapy.

D. Adult Skeletal Myoblasts (Satellite Cells)

The feasibility of implanting autologous myoblasts (satellite cells) harvested from the adult skeletal muscle has also been explored (Chiu, R C J, Zibaitis A, Kao L: Cellular cardiomyoplasty: Myocardial regeneration with satellite cell implantation. Ann. Thorac. Surg. 60:12-18, 1995). Using cell labeling techniques and phenotype specific antibodies, strong evidence has been presented that these myoblasts can undergo milieu-dependent transdifferentiation, and develop into striated muscle fibers with slow myosin heavy chains, as well as intercalated discs expressing Connexin-43. Taylor et al. confirmed that such engulfed satellite cells show ultrastructural features similar to immature cardiac myocytes, and when the implantation took place within a cryo-injured myocardium, cellular cardiomyoplasty could improve both the systolic and diastolic functions of such hearts (Taylor, D. A.; Atkins B. Z., Hungspreugs P., et al.: Regenerating functional myocardium: Improved performance after skeletal myoblast transplantation. Nat. Med. 1998; 4:929-933). Nevertheless, it would require sacrificing the patient's skeletal muscle, and the concern that the number of satellite cells in the skeletal muscle, as well as the satellite cells' mitotic potential may decrease with age. The optimal “conditions” for satellite cells to transdifferentiate into cardiomyocytes have not been clearly defined, and the molecular mechanisms of milieu-dependent differentiation remain unknown. Furthermore, it should be noted that the fundamental question of whether satellite cells can indeed transdifferentiate into true cardiomyocytes still remains controversial at present (Murry C E, Wiseman R W, Schwartz S M, Hauschka S D: Skeletal myoblast transplantation for repair of myocardial necrosis. J. Clin. Inves. 1996; 98:2512-2523).

Presently, it is widely known and accepted that the transplantation of an organ or cells from another individual in the same species (allo-transplantation), or from another species (xeno-transplantation) will result in rapid rejection by the host without immunosuppressive therapy for the host. This is due to the expression by the donor cells of specific allogeneic or xenogeneic antigens on their cell surface, which are recognized by the immune cells of the host as “foreign”, and elicit an immunological offensive to get rid of them. Although organ transplantation among individuals in the same species has been beneficial for many patients with immunosuppression, there is a severe limitation in donor organ supply, and immunosuppression has many side effects including increased risks for infection, malignancies and renal failure. In xeno-transplantation between different species, the problems are even more serious because the recipient has pre-formed circulating antibodies which can attack cells from different species immediately and violently, resulting in “hyper-acute rejection” which can kill the donor organ within hours if not in minutes. If such hyperacute rejection is prevented or reduced by complex procedures such as the use of transgenic animals or plasmorphoresis to remove pre-existing antibodies, the recipient will still undergo acute and chronic rejections which require significant immunosuppressive therapy. Accordingly, to date, xeno-transplantation is not generally accepted as a successful treatment option.

Liechty et al. have previously disclosed results of a xeno-tranplantation in which human mesenchymal stem cells were implanted in utero to fetal sheep. They report observing the survival of donor cells which underwent site specific differentiation (Liechty et al. Nature Medicine, Vol 6. No. 11, November 2000). However, it is well known that the immunological system is not fully developed in a fetus, and even in the newborn period in some species. Like the congenitally immunologically deficient animals, they can tolerate foreign cell implantation without rejection. To date, xeno-transplantation without immunosuppression in immunologically competent adults has not be achieved.

Thus, it would be further desirable to be provided with a means of inducing tissue cell growth and/or organ repair in vivo, without eliciting an immune response.

It would be further desirable to identify a suitable cell type for use in allo- and/or xeno-transplantation in immunologically competent, mature host subjects without immunosuppression to elicit tissue and/or organ repair therein.

It would be further desirable to be provided with means to perform myocardial implantation without eliciting an immune response and without sacrificing the patient's skeletal muscle.

It would be still further desirable to be provided with means to effect in vivo myocardial regeneration.

### SUMMARY OF THE INVENTION

One aim of the present invention is to provide means of inducing tissue cell growth and/or organ repair in vivo, without eliciting an immune response.

Another aim of the present invention is to provide a suitable cell type for use in allo- and/or xeno-transplantation in immunologically competent, mature host subjects without immunosuppression to elicit tissue and/or organ repair therein.

Another aim of the present invention is to provide means to perform myocardial implantation without eliciting an immune response and without sacrificing the patient's skeletal muscle.

Still another aim of the present invention is to use autologous MSCs in myocardial implantation to improve cardiac function.

A further aim of the present invention is to provide means for using a cell labeling technique to confirm the survival and differentiation of the implanted MSCs, and to identify MSCs phenotype by both morphology and molecular markers.

Another aim of the present invention is to provide means to examine the effects of the micro-environment of the implanted cells, on their differentiation and phenotype expression.

Another aim of the present invention is to provide means to examine the functional contribution of MSCs when they are implanted into an ischemic segment of the myocardium.

Another aim of the present invention is to provide a means for using marrow stem cells for improving cardiac function, wherein said marrow stem cells are introduced in situ into a myocardium.

In accordance with one aspect of the present invention, a method of inducing tissue cell growth and/or organ repair in vivo without eliciting an immune response is provided. The present invention employs undifferentiated stem cells to induce tissue cell growth and/organ repair in vivo without causing an immune response. The undifferentiated stem cells of the present invention may be autologous stem cells, stem cells obtained from a host of the same species as the recipient or stem cells obtained for a host of a species different from the recipient. Preferably, the undifferentiated stem cells of the present invention are bone marrow stroma cells (MSCs).

Recent studies in our lab as well as by others convincingly indicate that MSCs are adult stem cells capable of differentiating into all types of cells, ranging from cardiac and skeletal myocytes, nerve cells, liver and bone cells etc. MSCs appear to differentiate in situ, receiving signals from the microenvironment where they find themselves. We further found that these cells, when injected into the veins, can home-in and populate the host bone marrow, from where they can be recruited to the injured and malfunctioning organs throughout the bodies to participate in repairing the damaged tissues.

These observations lead us to a novel therapeutic strategy of the present invention.

The present invention also obviates the need for prior marrow ablation by radiotherapy or chemotherapy.

This is important since potential recipients with multiple organ failures are not ideal candidates for radical marrow ablation therapies, as the risks would be prohibitive.

In patients who suffer from single or multiple organ failures from various causes (trauma, infection, degenerative processes etc), it has been shown that the patients' own MSCs are recruited to the injury site(s), and participate in the process of repairing the damage. In accordance with a further aspect of the present invention, we propose that this normal process can be therapeutically augmented by the injection, either systematically by transvenous infusions, or locally by topical administration, of MSCs harvested from another subject of same (allograft) or different species (xenograft). One advantage of this approach compared to the use of autologous MSCs is that the lag time between harvesting of MSCs to their availability for transplantation, required to culture and expand the MSC population in vitro, can be eliminated.

According to the present invention, MSCs are determined to traffic through the circulatory system to the injured heart, and are capable of forming cardiomyocytes and other types of cells, depending on the specific microenvironment. In addition, endothelial progenitor cells in the MSCs population may be involved in the post-infarction neovascularization process. As a result, MSCs display myocardial cell differentiation properties in vivo and provide a promising therapeutic use in improving myocardial healing following infarction. Labeled cardiac myocytes and fibers were present in the implant site, which exhibited positive immunohistochemical stains in normal or immature cardiac myocytes. These findings indicate that it is possible to use autologous marrow stroma cells (MSCs) in transplantation for myocardial regeneration in animals and humans.

In addition, the present invention has application in the area of experimental research, where by MSCs may be employed in a variety of in vivo animal models to further study the influence of the micro-environments on stem cell differentiation, and on cellular signaling mechanisms.

Further echocardiographic studies are expected to demonstrate improved systolic thickening of the ischemic ventricular wall segment, and reduced ventricular size and remodeling, as had been reported following the implantation of other donor cells.

Future studies include clinical trails and mechanistic investigations employing the teachings of the present invention. Minimal medical and ethical difficulties are expected for clinical trials encompassing the present invention for the numerous reasons herein described. It is fully contemplated that the findings of the present invention will offer a valuable basis to pursue such scientific knowledge. For example, a comparison of the differentiation of labeled cells implanted at the center, and at the periphery of the infarcts will be made. In addition the microenvironments responsible for MSC differentiation in the myocardium will be further explored. Differentiation may be facilitated at the peripheral border zone, if direct cell-to-cell contact is an important signaling mechanism for such differentiation to take place. The role of cytokines and other growth factors will also be examined in the future. Further still, methodologies for transplanting autologous MSCs in patients to improve cardiac function will be optimized in future studies.

In accordance with another aspect of the present invention, there is provided a method for performing bone marrow transplantation using the stromal cells which does not require prior marrow ablation by radiotherapy or chemotherapy. This is important since the potential recipients with multiple organ failures are not candidates for radical marrow ablation therapies, as the risks would be prohibitive.

In accordance with the present invention there is further provided a new therapy to repair damaged organs in the body, by using bone marrow stromal cells of another individual in the same species, or from another species, without immunosuppressing the host subject. Since harvesting, culturing and expanding the numbers of marrow stem cells take time, it is highly valuable to have such stem cells available instantly in critically ill patients. Allogeneic and xenogeneic marrow stromal cells could be isolated under sterile conditions, culture expanded in vitro, ready to be shipped for patient application. We think there is a commercial value for such a product which could be useful in a vast number of patients.

In accordance with an aspect of the present invention, bone marrow stoma cells (MSCs) were employed in in vivo myocardium implantation and found to effect growth of new muscle fibers and improve overall cardiac function.

In accordance with another aspect of the present invention, there is provided a use of MSCs for improving cardiac function wherein said MSCs are transplanted in situ into a myocardium, and differentiate into cardiomyocytes, fibroblasts and endothelial cells.

In accordance with the present invention, there is provided a method of improving cardiac function in a patient with heart failure without eliciting an immune response and without sacrificing the patient's skeletal muscle; which comprises the step of transplanting bone marrow stroma cells (MSCs) into said patient's myocardium to grow new muscle fibers.

The method may further comprise the step of using a cell labeling technique to confirm survival and differentiation of implanted MSCs, and to identify said MSCs phenotype by both morphology and molecular markers.

The method may further comprise examining the effects of the micro-environment of implanted MSCs on their differentiation and phenotype expression.

The method may further comprise examining functional contribution of MSCs implanted into an ischemic segment of the myocardium.

The transplanting may be effected in the myocardium in situ, in the coronary artery or using a catheter from within the ventricular cavity.

The transplanting may also be effected in association with angiogenesis factors.

The bone marrow stroma cells (MSCs) of the present invention may be autologous MSCs, MSCs transplanted from a host of the same species (allograft)or MSCs transplanted from a host of a different species (xenograft).

In accordance with a further aspect of the present invention, there is provided a method of inducing tissue cell growth and/or organ repair in vivo without eliciting an immune response; which comprises the step of transplanting undifferentiated bone marrow stroma cells (MSCs) from a host of one species into a recipient of the same species. In accordance with a yet further aspect of the present invention, there is provided a method of inducing tissue cell growth and/or organ repair in vivo without eliciting an immune response; which comprises the step of xeno-transplanting undifferentiated bone marrow stroma cells (MSCs) from a first species into a second species.

In accordance with a yet further aspect of the present invention, there is provided a use of marrow stroma cells (MSCs) from a host of one species into a recipient of the same species for improving tissue cell growth and/or organ repair, wherein said marrow stem cells are introduced in situ into said recipient.

In accordance with a yet further aspect of the present invention, there is provided a use of marrow stroma cells (MSCS) from a first species into a second species for improving tissue cell growth and/or organ repair, wherein said marrow stem cells are introduced in situ into said second species.

In accordance with still a further aspect of the present invention, there is provided a method of treating tissue and/or organ damage in vivo, said method comprising: (a) retrieving bone marrow from a host of one species; (b) isolating marrow stroma cells from said bone marrow said host; (c) expanding said marrow stroma cells in culture; and (d) transplanting said marrow stroma cells into a recipient of the same species.

In accordance with a yet another aspect of the present invention, there is provided a use of marrow stroma cells obtained from a host for examining the effects of a tissue- or organ-specific microenvironment on marrow stroma cell differentiation, wherein said marrow stroma cells are introduced in situ into a recipient, said recipient of an animal model.

In accordance with still another aspect of the present invention, there is provided a method of treating cardiac failure in a patient, said method comprising: (a) retrieving bone marrow from one of a host or said patient suffering from cardiac failure; (b) isolating morrow stroma cells from said bone marrow; (c) expanding said marrow stroma cells in culture; (d) treating said marrow stroma cells in culture to induce a cell phenotype capable of enhancing cardiac function; and (e) transplanting said marrow stroma cells into a myocardium of said patient suffering from cardiac failure.

The bone marrow stroma cells of the present invention may be treated to induce cell differentiation to produce a variety of cell types, including, without limitation cardiomyocytes, fibroblasts, endothelial cells, skeletal myocytes, nerve cells, liver and bone cells, osteoblasts, chondroblasts, adipocytes, myoblasts and angiogenic cells.

The host of the bone marrow cells employed in accordance with the present invention may be of the same species as the recipient or another species.

For the purpose of the present invention the term “host” is intended to mean a donor having in situ cells of interest which may be obtained therefrom for the purposes of this invention.

Recently, preliminary studies were carried out involving the transplantation of bone marrow stroma cells (MSCs) from one member of a species to another member of the same species (allo-transplantation) and transplantation from a member of one species to a member of a different species (xeno-transplantation) to elicit tissue cell growth and/or organ repair.

In accordance with the present invention, these studies, as herein described, have shown that bone marrow stromal cells (MSCs) harvested from an animal can be transplanted into another animal of same (i.e. “allograft”) or different (i.e. xenograft) species successfully without immunosuppression of the recipient animals to prevent rejection. Apparently stem cells such as MSCs can escape the immune surveillance of the host animals, either by interacting and suppressing the immune system of the host, or by expressing the host histocompatibility antigen markers during their differentiation to avoid rejection by the competent host immune system. In either case, they do not require immunosuppressive therapy, which often carries significant side effects, to achieve long term engraftment.

In accordance with the present invention, the rationale for using donor cells in a recipient from a host of the same species or from a host of a different species, without eliciting an immune response to improve tissue cell growth and/or induce organ repair will be evident.

### BRIEF DESCRIPTION OF THE DRAWINGS

FIG. 1 illustrates four days after implantation of MSC into the myocardium; Top panel: Hematoxylin and eosin stain; Lower panel: Fluorescent microscopy picture, showing MSCs labeled with DAPI;

FIG. 2 illustrates four weeks post-implantation;

FIG. 3 illustrates specimen four weeks after MSC implantation into the myocardium; Top panel: Hematoxylin and eosin stain; Lower panel: Fluorescent cells originated from MSCs labeled with DAPI in vitro;

FIG. 4 illustrates rat MSCs morphology in culture, and in particular, a phase contrast photomicrograph of twice-passaged culture of MSCs just before implantation. Most adherent MSCs are practically fibroblastic in morphology. Scale bar represents 30 μm;

FIG. 5 illustrates histochemical staining for β-galactosidase activity of rat MSCs in culture. The transfected MSCs showed clear staining for β-galactosidase activity. Transfection efficiency of the MSCs was approximately 100%. Scale bar represents 60 μm;

FIG. 6 illustrates β-gal positive cells trapped within a coronary capillary immediately after MSCs injection. Staining for β-galactosidase activity followed by H & E stain. Arrow, a capillary endothelial cell. Scale bar represents 15 μm;

FIG. 7 illustrates β-gal positive cells with i morphology outside infarct scar 4 weeks after MSCs injection. Staining for β-galactosidase activity followed by H & E stain. Arrows, intercalated disk-like structure. Arrowhead, the nucleus of a β-gal positive cell. Scale bar represents 15 μm;

FIG. 8 illustrates β-gal positive cells with fibroblast-like morphology in the myocardial scar 4 weeks after MSCs injection. Staining for β-galactosidase activity followed by H & E stain. Arrows, β-gal positive cells. Scale bar represents 1.5 mm. (Inset) Higher magnification of the area in square. Scale bar represents 30 μm;

FIG. 9 illustrates β-gal positive cells incorporated into endocardium 4 weeks after MSCs injection. Staining for β-galactosidase activity followed by Eosin stain. Arrow, endocardium. Scale bar represents 15 μm;

FIGS. 10A & 10B illustrate β-gal positive cells incorporated into coronary capillaries 4 weeks after MSCs injection. Staining for β-galactosidase activity followed by H & E stain. A. Outside the infarct scar. Arrow, a capillary with β-gal positive cells in the subendocardial fibrosis area. Arrowhead, normal myocardium. Asterisk, endoventricular space. Scale bar represents 600 μm. (Inset) Higher magnification. Arrow, the cross section of the same capillary with β-gal positive cells. Scale bar represents 15 μm. B. In the infarct scar. Arrow, the oblique section of a capillary with β-gal positive cells. Scale bar represents 15 μm;

FIG. 11 illustrates histochemical stain for connexin-43 in the intercalated discs (arrows), demonstrating the presence of gap junction unique to myocardium in the labeled (blue) myocytes;

FIG. 12 illustrates bone marrow taken from a rat 24 hours after transplantation which includes labeled marrow stromal cells;

FIG. 13 & 14 illustrate labeled marrow stromal cells in the scar and border zone of a myocardial infarction;

FIG. 15 illustrates a histological section of the implantation site of a rat heart implanted with pig bone marrow cells;

FIG. 16 illustrates another histological section of the implantation site of a rat heart implanted with pig bone marrow cells observed under fluorescent microscopy showing fluorescence of the cardiac muscles at the implantation site; and

FIG. 17 illustrates an immunohistochemical stain of a section of a rat heart implanted with pig bone marrow cells using antibodies against troponin 1-C.

### DETAILED DESCRIPTION OF THE PRESENT INVENTION

In accordance with the present invention, there is provided means of inducing tissue cell growth and/or organ repair in vivo, without eliciting an immune response.

The present invention further provides a means of using a suitable cell type in allo- and/or xeno-transplantation in immunologically competent, mature host subjects without immunosuppression to elicit tissue and/or organ repair therein.

Further, the present invention provides means of improving tissue cell growth and/or organ repair in vivo without eliciting an immune response.

The present invention is exemplified in connection with bone marrow stromal cells, however, it is not intended to be limited thereto.

In accordance with the present invention, there is also provided means to perform myocardial implantation without eliciting an immune response and without sacrificing the patient's skeletal muscle.

It is widely accepted that the embryonal stem cells are multipotent because they have to develop into a fetus and then an adult individual composed of numerous types of cells. However, the reasoning for a fully grown individual to continue to possess cells with the potential to develop into many different phenotypes was not previously known or investigated.

In accordance with the present invention it is suggested that these cells are in reserve in fully grown individuals to be recruited by various organs for growth and repair. Signaling mechanisms are believed to be at work to induce these adult stem cells to migrate from the bone marrow, via the blood stream to the injured organ, where they undergo site specific differentiation to replace injured cells in adult individuals.

In accordance with the present invention, we have shown that marrow stromal cells can be infused intravenously and they will home into and reside in the bone marrow where they serve as a reserve for organ repair. From there they can be recruited to various organs and undergo site specific differentiation to repair damaged tissues. Thus, according to one aspect of the present invention, in patients who suffer or are likely to suffer multiple organ failures, we propose to obtain autologous marrow stromal cells, culture and vastly expand the population of the cells in vitro, and infuse them back to the patient, thereby augmenting the repair capability for damaged organs. This infusion of autologous marrow stromal cells can further serve to prevent or ameliorate multiple organ failure and death. Furthermore, according to another aspect of the present invention, it is shown that it is possible to use allogeneic or xenogeneic marrow stromal cells as donors for marrow stromal cell transplantation to elicit tissue cell growth and/or organ repair.

The allo- or xeno-transplant donor MSCs can be prepared ahead of time and maintained in culture for immediate use, in a way similar to getting blood from blood bank for transfusion immediately into patients in hemorrhagic shock. Eliminating the lag time with this technique could save gravely ill patients who could not wait for their own MSCs to be harvested, cultured and expanded over many days or weeks. Since these MSCs are not immunologically detected and rejected by the host, there is no need for “type and cross matching” them, as is required for blood transfusions.

According to this aspect of the present invention, a new therapy to repair damaged organs in the body, by using bone marrow stromal cells of another individual in the same species (allograft), or from another species (xenograft), without immunosuppressing the host subject is provided. Since harvesting, culturing and expanding the numbers of marrow stem cells take time, it is highly valuable to have such stem cells available instantly in critically ill patients. Allogeneic and xenogeneic marrow stromal cells could be isolated under sterile conditions, culture expanded in vitro, ready to be shipped for patient application. We think there is a commercial value for such a product which could be useful in a vast number of patients.

The ability to regenerate a functioning cardiac muscle in patients with heart failure, who have lost a significant amount of native cardiac muscle fibers through ischemic necrosis and apoptosis, has enormous therapeutic potential in the treatment of heart failure. Autologous marrow stroma cells (MSCs) have been identified as target donor cells for eliciting mycocardial regeneration in vivo. Using autologous MSCs as donor cells for cell transplant therapy has a number of important advantages. In particular, by using autologous MSCs in cell transplant therapy, the need for fetal tissue and its ethical and legal controversies can be avoided, while also avoiding the need for immunosuppression. Unlike using modified cardiac myocytes or established cell line myoblasts, the danger of oncogenicity can be diminished. In addition, using autologous skeletal myoblasts would require the sacrifice of a patient's skeletal muscle, which is irreplaceable. In contrast, bone marrow puncture, a routine clinical procedure, can be repeated to harvest MSCs for more than one occasion. The procedure is also much less invasive, and can be easily performed in patients under local anesthesia, in contrast to the excision of a major muscle mass, such as the latissimus dorsi muscle in order to harvest satellite cells. The latter procedure would likely require general anesthesia. Considering that such patients will be already suffering from severe heart failure, invasiveness of the procedure required for donor cell harvesting could be an important clinical consideration. The cell implantation procedure can be combined with other surgical operations, such as coronary bypass surgery, or by minimally invasive surgical techniques or by transvenous catheter injections.

Possible problems include false positive results associated with some cell labeling techniques as well as the interpretation of phenotype specificity associated with immunohistochemical findings. For example, a myosinslow molecule may be detected both in Type I skeletal muscle fibers as well as in cardiac myocytes, and Connexin 43 may be expressed in immature myoblasts. Nevertheless, by employing several different cell labeling techniques and immunostain antibodies, correlating with histological and ultrastructural examinations, much of these uncertainties may be addressed.

Previous proposals to use certain sources of donor cells for cellular cardiomyoplasty are not ideal for clinical application, owing primarily to the need for fetal tissue, and/or to the need for immunosuppression (Soonpaa M. H., Koh G. Y., Klug M. G., Field L. J. Formation of nascent intercalated disks between grafted fetal cardiomyocytes and host myocardium. Science 1994;264:98-101; Li R. K., Jia Z. Q., Weisel R. D., Mickle D. A. G., Zhang J., Mohabeer M. K., et al. Cardiomyocytes transplantation improves heart function. Ann Thorac Surg 1996;62:654-61; Taylor D. A., Atkins B. Z., Hungspreugs P., Jones T. R., Reedy M. C., Hutcheson K. A., et al. Regenerating functional myocardium: Improved performance after skeletal myoblast transplantation. Nature Medicine 1998;4:929-33; Klug M. G., Soonpaa M. H., Koh G. Y., Field L. J. Genetically selected cardiomyocytes from differentiating embryonic stem cells form stable intracardiac grafts. J. Clin. Invest. 1996; 98:216-24).

The present invention demonstrates that the in vivo myocardial environment can support the growth and induce the cardiomyogenic differentiation of MSCs. Compared to other cell sources, the present invention illustrates the advantages in the clinical use of MSCs for cellular cardiomyoplasty.

Grafting of cells into the myocardium requires some form of delivery system. The choice for the routes of cell implantation may depend on the pathology of the heart. Up to now, most of studies in the field of cellular cardiomyoplasty were performed by direct injection of various cells into the myocardium. Though implanted cells may have the ability of migration along the ventricular surface of heart (Connold A. L., Frischknecht R, Dimitrakos M, Vrbova G. The survival of embryonic cardiomyocytes transplanted into damaged host rat myocardium. J Muscle Res Cell Motil 1997;18:63-70), this procedure covers only a limited field and may require multiple injections, either through epicardium, or via the endocardium. Coronary arterial delivery of donor cells to the myocardium possesses theoretical advantages, at least for certain types of heart failures. Thus, in order to optimize the strategy for cell implantation, the feasibility of delivering MSCs by selective infusion into the coronary circulation was evaluated.

In accordance with an aspect of the present invention MSCs are infused into coronary artery and appear to repopulate the heart. Further, signals originating in the cardiac milieu appear to modify the developmental program of the infused MSCs. Studies conducted in accordance with the present invention further confirm the residence of MSCs outside of the capillary bed, and illustrate the structural interactions between the host myocardial tissues and the implanted MSCs.

To trace the fate of infused MSC according to an embodiment of the present invention, MSCs were retrovirally transfected with β-gal reporter gene for cell labeling. Compared with other virus-based gene transfer, retrovirus has less immunological response and longer gene expression (Onifer S M, White L A, Whittemore S R, Holets V R. In vitro labeling strategies for identifying primary neural tissue and a neuronal cell line after transplantation in the CNS. Cell Transplantation 1993;2:131-149). Using current transfection model, the transfection efficiency in culture is approximately 100% without obvious adverse effect on the cell growth. Our in vivo control studies, including infusion of MSCs culture supernatant, nontransfected MSCs or lysed transfected MSCs while performing the same read-out, confirmed the specificity of this cell labeling technique.

Immediately after the infusion, the MSCs were trapped within the coronary capillaries in the non-infarct area. The reason MSCs could not be found in the infarct scar at this time may be related to the complete occlusion of involved coronary artery (Left coronary artery). However, 4 weeks after infusion, the MSCs could be found both in the infarcted scar and in non-infarct area outside the vascular structure. The mechanism of the translocation of MSCs from the vascular lumen into the myocardial interstitium is unknown. MSCs may migrate out of the vasculature and move from the non-infarct area to the infarction scar. The other possible explanation is that scar of myocardial infarction is not a completely dead tissue. The reason why MSCs can only be found in 60% of rats (6 out of 10) 4 weeks after injection may be related to the infusion technique. By using this model of briefly clamping the ascending aorta distally, some cells may have leaked out of the puncture hole on the ascending aorta. Others may escape through the blood stream to distal organs and tissues.

MSCs in different myocardial microenvironments clearly have different fates. In the non-infarct area, they express the phenotypes of normal cardiomyocytes and connected with surrounding host cardiomyocytes by intercalated disk-like structure. In the infarct scar, they appear primarily fibroblast-like. This homing ability and the capability to acquire the phenotypes of different target tissues suggest that the microenvironment plays a significant role for the differentiation of these cells.

According to the present invention, in contrast to myocardial scar tissue, the normal myocardial microenvironment appears to enable newly arrived cells to be exposed, in an appropriate sequential manner, to various cardiomyogenic specific growth factors and differentiation molecules, such that the infused MSCs could develop into fully mature cardiomyocytes. The fibroblast-like MSCs seen in the infarction scar could have differentiated into primary fibroblast, which are mature mesenchymal cells or they could still maintain the multipotent differentiation ability for future maturation.

The studies of the present invention revealed that some β-gal positive donor cells differentiate into endothelium, which was incorporated into capillaries in the infarct and non-infarct areas. Accordingly, these marrow-derived endothelial progenitor cells are likely to be involved in the angiogenesis and vasculogenesis in the remodeling process of myocardial infarction. Thus further suggesting the potential of use of MSCs for implantation into the myocardium to improve heart function.

In addition, it has been shown that some cells in MSCs culture are positive for Factor VIII-associated antigen (Singer J W, Charbond P, Keating A, Nemunaitis J, Raugi G, Wight T N, et al. Simian virus-40 transformed adherent cells from human long-term marrow cultures: Clone cells produced with “stromal” and hematopoietic characteristics. Blood 1987;70:464-474), suggesting an endothelial origin. Shi et. al suggested that a subset of cells localized in the bone marrow could be mobilized to the peripheral circulation and colonize endothelial flow surfaces of vascular prostheses (Shi B Q, Rafii S, Wu M H D, Wijelath E S, Yu C, Ishida A, et al. Evidence for circulating bone marrow-derived endothelial cells. Blood 1998;92:362-367).

Both localized site-specific and global delivery of autologous MSCs may be of potential therapeutic benefit in view of different cardiac pathology. For example, intra-coronary delivery of MSCs may be more suitable for the treatment of heart failure due to diffuse cardiomyopathy. Furthermore, the present invention demonstrates that when expanded marrow-derived stromal cells are delivered to the coronary circulation of an infarcted heart, they are capable of populating the heart and differentiating along several lineages including cardiomyocytes, fibroblast and endothelial cells.

These findings, in accordance with an aspect of the present invention suggest that infracted heart muscle can signal mobilization of MSCs to enter circulation, and reach the coronary artery, where they may participate in myocyte replenishment, reactive fibrosis and scar formation as well as angiogenesis in the post-infarct pathophysiological remodeling process, involving both the infarcted segment and the remote non-infarcted areas. Additional studies in accordance with the present invention will further elucidate the role of MSCs in myocardial infarction and the clinical applications of MSCs implantation. It is fully contemplated that the findings presented in accordance with the present invention will enable therapeutic modulation of the remodeling process after myocardial infarction, in both animals and humans.

The present invention will be more readily understood by referring to the following examples which are given to illustrate the invention rather than to limit its scope.

### EXAMPLE I

### Preliminary Studies

In a preliminary study, isogenic rats were used as donors and recipients, since as in the autotransplants, this model obviates the need for immunosuppression. Lewis rats weighting 175 to 200 grams were used in all experiments. As will be described in detail below, femoral and tibial bones were explanted from the donor rats and used as the source for bone marrow stroma cells. Details of isolation, plating and passaging techniques for MSC will also be described below. The MSCs were expanded in culture for 2 to 3 weeks and labeled with DAPI (4′, 6-diamidino-2-phenylindole) before transplanting them into the lateral wall of the left ventricle of the recipient rat hearts. This was accomplished by direct injection of MSC suspension using a 28-gauge needle. At different time intervals ranging from 2 days to 4 weeks following the implantation, the hearts were harvested and studied histologically. The hearts at the implant sites were sectioned serially, and stained with hematoxylin and eosin. Slides of the adjacent section to the above were examined under fluorescent microscopy to identify DAPI, which upon binding to the DNA of the MSCs prior to implantation, can be recognized as fluorescent positive cells.

The sections (6 μm/thick) were also examined immunohistochemically using antibodies against myosinslow molecules. Other serial sections from these specimens are currently undergoing studies using antibodies against the cardiac gap junctional protein, Connexin-43, and other phenotype specific antibodies, for immunolabeling and staining.

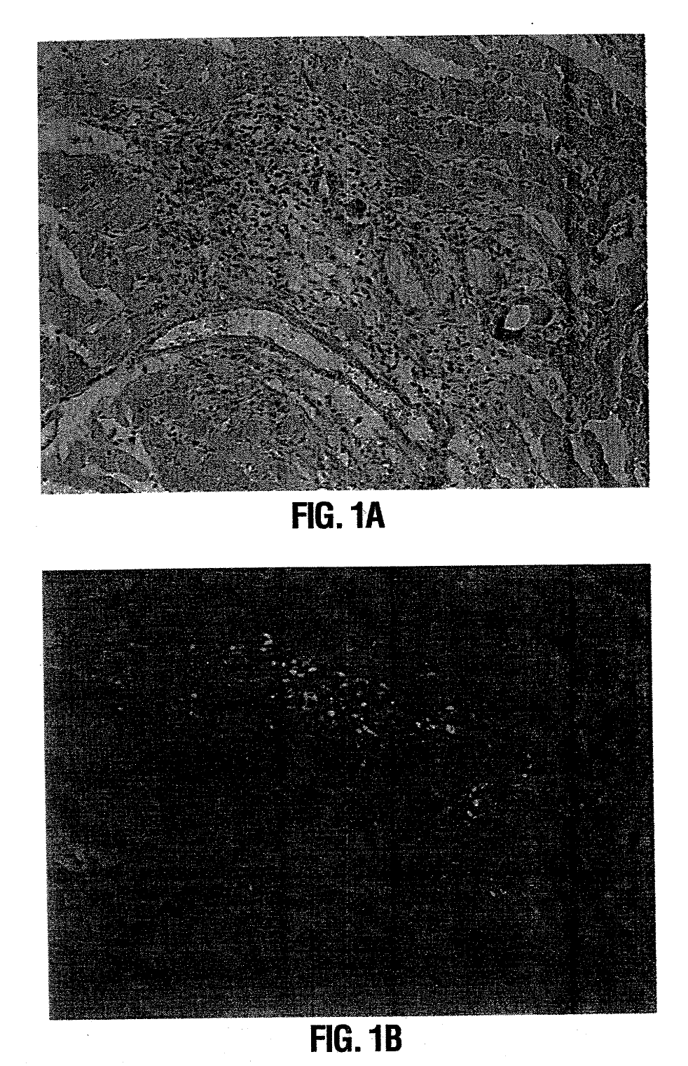

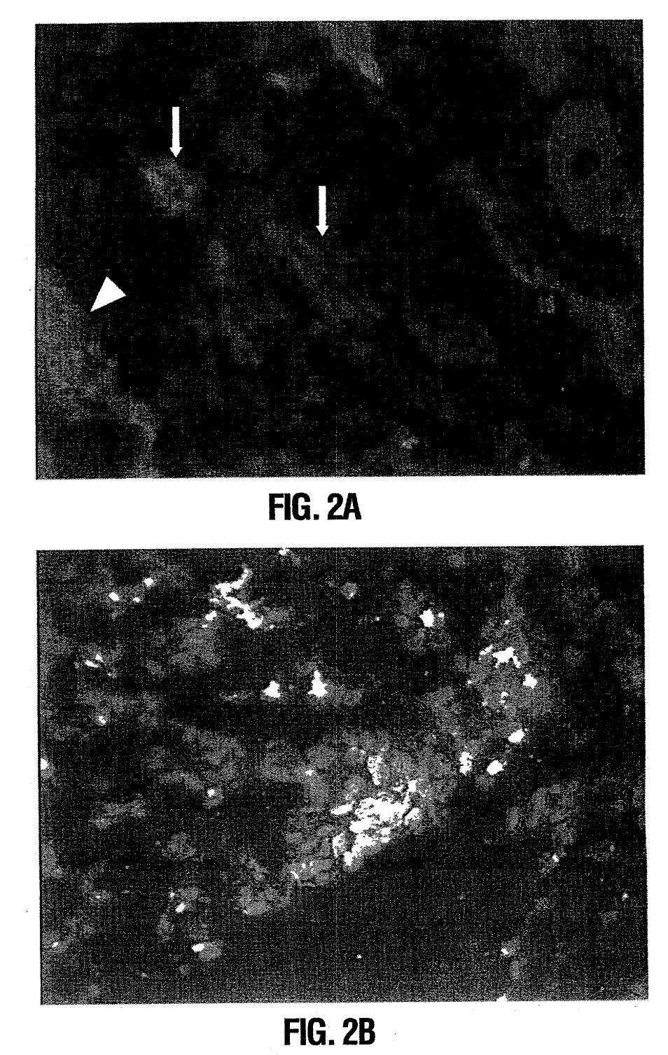

FIG. 1 illustrates a microphotograph obtained 4 days after implantation. The implant site shows a needle track created during the process of injecting the MSCs, with some inflammatory response and fibrosis within the needle track. Fluorescent microscopic examination demonstrated the presence of the labeled MSCs implanted. FIG. 2 shows labeled cells in the injection site, immunolabeled with myosin-slow antibodies which stains red; DAPI labeled fluorescent cells within needle track made during implantation; and immunohistochemical stain with antibody against slow myosin heavy chain, which shows red color. The triangle marker points to the native cardiac myocyte, and arrows point to the appearance of myosin molecules in the cytoplasm of implanted cells.

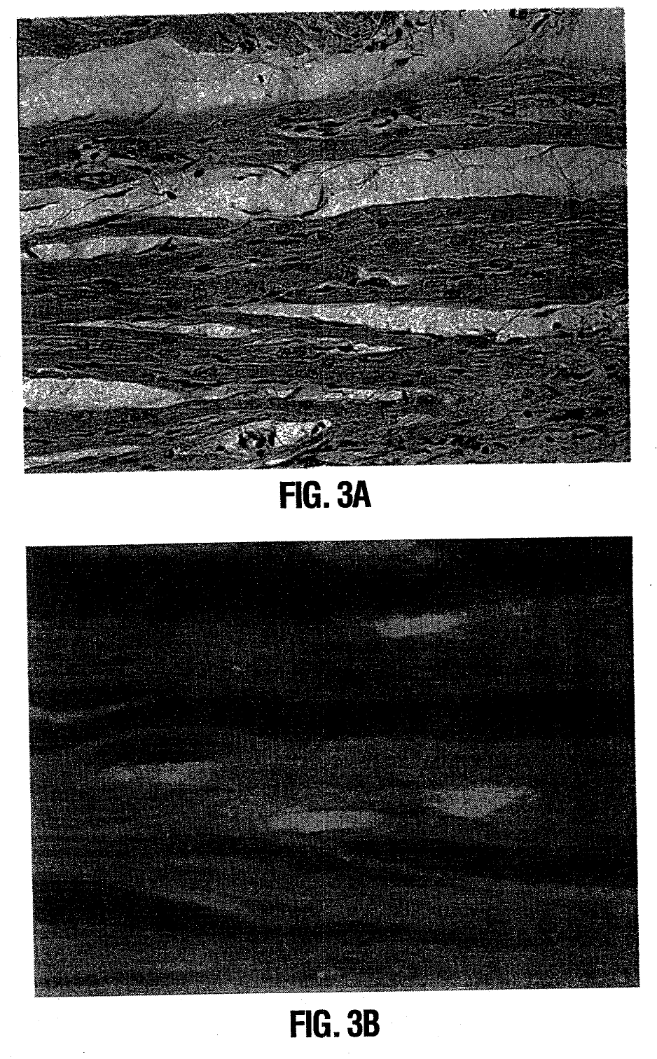

The deep red color shows myosin-slow heavy chains in the native muscle (triangle), whereas the red stain adjacent to the labeled cells suggest the synthesis of myosin-slow molecules in the implanted cells (arrows). FIG. 3 is a photograph taken a short distance away from the implant needle track, showing migrated or infiltrated MSCs appearing to have differentiated fully and were incorporated into cardiac muscle fibers, morphologically indistinguishable with the native myocardium. Clear labeling of these cells can be demonstrated under fluorescent microscopy. Morphologically, they appear identical to the native myocardial fibers. Photographs were taken from the myocardium adjacent to the needle track, where the implanted cells had migrated or infiltrated.

Additional studies are described below to confirm these findings, as well as to further elucidate the phenotypes of the new muscle using additional specific antibodies. These preliminary studies, however, clearly demonstrated various experimental techniques, ranging from isolation, culture and identification of MSCs, as well as implanting of these cells to the rat hearts with virtually no mortality.

Experimental Protocol: The Rationale for Experimental Models

In clinical application of one embodiment of the present invention, use of autologous MSCs for cardiac implantation to improve cardiac function is particularly advantageous in that it avoids the need for immunosuppression. Relatively inexpensive isogenic Lewis rats were chosen for the preliminary study. The preliminary investigation described above indicates that this is a useful and reliable model, with little operative mortality for cardiac cell implantation resulting. Furthermore, the capability to perform coronary artery ligation and sequential echocardiographic studies in such animals is confirmed and the feasibility of proposed experiments in accordance with the present invention is supported.

Experimental Design

For each rat receiving cardiac cell implant (experimental animal), there will be an isogenic rat to serve as the donor of bone marrow stroma cells. Another group of sham operated rats (controls) will undergo identical surgical procedures as the experimental animals, but will receive injection of cell culture media without MSCs.

a) Donor rats will be sacrificed, and their femoral and tibial bones will be used to isolate, select and culture MSCs in vitro for 2 weeks using the technique described below. Then the cells will be collected, labeled and injected into the myocardium of the experimental recipient rats.

b) Cell implant recipient (experimental) rats: The prospective future recipients for cell implant will undergo thoracotomy and ligation of the anterior descending coronary artery (see below). The chest will be closed and the animals monitored with weekly echocardiographic studies to observe changes in the ventricular wall motion of the ischemic zone for 2 weeks. In the second thoracotomy, these rats will receive injection of isogenic MSCs cultured and labeled in vitro. The injections will be made through a 26-gauge needle into the anterolateral wall of the left ventricle, both at the center of the infarct zone, as well as at the peripheral border zone between the infarcted and non-infarcted cardiac muscles. Following the implant procedure, the chest will be closed and studied weekly using echocardiography as described below. They will be sacrificed after cell implantation at an interval of 4 days, 2 weeks, 4 weeks, and 3 months, with a sample size of 10 rats each. Frozen sections will be made serially through the implant site, at a thickness of approximately 6 μm each, and slides will be process for fluorescent microscopic, histological, immunohistochemical and electron microscopic studies as described in detail below.

c) Sham operated (control) rats: This group will undergo coronary artery ligation and cardiac implantation procedures exactly as described above for the experimental group. However, instead of receiving cultured and labeled MSCs, they will receive the same volume of culture media only. They will be harvested at the same time intervals and studied in the same manner as described for the experimental animals.

Sample Size

The sample size of 10 was based on our preliminary study as this is a highly reliable model that resulted in minimal operative mortality. In the future, wall motion studies will be performed to calculate the sample size required.

All animals will receive humane care and all experiments will be performed according to the “Guidelines to the Care and Use of Experimental Animals” of the Canadian Council on Animal Care.

Isolation, Plating and Passaging Techniques of Bone Marrow Stroma Cells

Isolation and primary culture of MSCs will be performed according to Caplan's method. After overdose with pentobarbital (100 mg/kg given intraperitoneally) (MTC Pharmaceuticals, Cambridge, Ontario), the femoral and tibial bones of the donor Lewis rats (weighing 175 to 200 grams) will be collected and the adherent soft tissue removed. Meticulous dissection of the long bones will be carried out in order to remove soft tissue to ensure that myogenic precursors are not carried into the bone marrow preparation. Both ends of the bones will be cut away from the diaphysis with bone scissors. The bone marrow plugs will be hydrostatically expelled from the bones by insertion of 18-gauge needles fastened to 10 ml syringes filled with complete medium; the needles are inserted into the distal ends of the femoral and proximal ends of the tibial bones, and the marrow plugs expelled from the opposite ends. The marrow plugs are disaggregated by sequential passage through 18-gauge, 20-gauge and 22-gauge needles and these dispersed cells are centrifuged and resuspended twice in complete medium. Cell viability is assessed by the trypan blue exclusion test. After the cells are counted in a hemocytometer, 5×107 cells in 7-10 ml of complete medium are to be introduced into 60 mm polystyrene tissue culture dishes (Corning, Inc., Corning, N.Y.), which are coated in advance with a layer of laminin (Sigma) to promote marrow stroma cell adherence. Three days later, the medium is changed and the nonadherent cells discarded. The medium is completely replaced every 3 days. In approximately 10 days after seeding, the dishes will become nearly confluent and the adherent cells can be released from the dishes with 0.25% trypsin in 1 mmol/L sodium ethylenediaminetetraacetic acid (Gibco Laboratories, Grand Island, N.Y.), split 1:2, and seeded onto fresh plates. After these twice passaged cells become nearly confluent, they can be harvested and used for implantation experiments described below after being labeled with DAPI. The “complete medium” mentioned above for our culture consists of Dulbecco's modified Eagle's medium (DMEM, Gibco Laboratories) containing selected lots of 10% fetal calf serum (FCS; JR Scientific Inc., Woodland, Calif.), and antibiotics (Gibco Laboratories; penicillin G, 100 U/ml; streptomycin 100 μg/ml, amphotericin B 0.25 μg/ml) at 37° C. in a humidified atmosphere of 5% C02.

Bone Marrow Stroma Cell Labeling

Although in this preliminary studies, we have used DAPI for cell labeling, retroviral vectors as the tool for cell labeling may also be employed. Likewise, other known techniques for cell labeling may be employed in accordance with the present invention. Retroviral vectors permit insertion of foreign synthetic genetic information in target cells. Reporter genes such as β-galactosidase, can therefore be stably integrated in chromosomal DNA. Expression of these transgenes permits ambiguous identification, by classic histochemical or fluorescence microscopy, of gene modified cells. As an example, retroviral labeling of hematopoietic stem cells is routinely carried out in the study of bone marrow transplantation in rodents. Undifferentiated (gene labeled) progeny cells of a widely different phenotype (lymphocytes, granulocytes, platelets, red blood cells) are readily detectable up to one year after bone marrow transplantation. We propose that retroviral labeled stroma cells that survive, proliferate or differentiate following cardiac implantation, will preserve and express the transgene as is classically observed in animals transplanted with retrovirus labeled hematopoietic stem cells. With this technique, important questions regarding bio-distribution of transplanted stroma cells can be addressed. Unambiguous identification of “labeled stroma cells”, as well as their differentiated progeny, local and distant to the site of implantation in the injured heart, will become possible. Identification of other “unexpected” progeny cells, such as endothelial cells, interstitial cells, possibly others, will also be feasible. Furthermore, in accordance with an embodiment of the present invention sensitive PCR-based techniques were employed to detect as little as 1 to 100,000 transgene positive cells from tissue DNA extracts.

According to an embodiment of the present invention cultured primary marrow stroma cells were readily transduced with synthetic retroviral vectors. In addition, high efficiency gene transfer in cultured stroma cells may also be employed and genetically labeled cells can be expanded in vitro for up to 3 to 4 weeks without loss of reporter gene expression (in this case the green fluorescent protein) . We have on hand a high titer VSV-G pseudotyped β-gal retroviral producer. We propose that transplanted stroma cells genetically labeled with a β-galactosidase retrovector will be readily identified by classic X-Gal staining of cardiac tissue sections.

Coronary Artery Ligation Model in Rats

The recipient rats, both experimental and control, are isogenic Lewis rats weighing 175 to 200 grams. Anesthesia is induced and maintained with isoflurane (MTC Pharmaceuticals). The animals are intubated with an 18-gauge intravenous catheter and connected to a Harvard rodent ventilator (Harvard Apparatus Co., Inc., South Natick, Mass.) at 85 breaths per minute. The heart is exposed via a 1.5 cm left thoracotomy incision. Under direct vision, using a 5-0 prolene suture, the anterior descending coronary artery which is visible in the epicardium is ligated proximally. The thoracotomy is closed with 4-0 monofilament sutures. The muscle and skin layers are closed with 4-0 absorbable sutures and the animals are returned to their cages with filter tops. After the learning curve, the operative mortality is virtually nil.

Cell Transplantation into the Rat Heart

The recipient rats which underwent coronary artery ligation 2 weeks previously will undergo a second operation. Anesthesia and thoracotomy will be performed in the manner described above. Under direct vision, the MSC suspension is injected into the lateral wall of the left ventricle with a 20-gauge needle, both at the center of the ischemic segment of the myocardium, and at the border zone at the junction between the infarcted and normal myocardium. The thoracotomy closure and post-operative care are similar to that described above, and the animals sacrificed at intervals after this procedure, as stated earlier.

The sham operated control rats will undergo an identical procedure as described for cell implantation in the experimental animals. The only difference is that instead of injecting cultured MSCs, an identical volume of culture media (component described above) will be injected.

Echocardiographic Studies on Wall Motion

Transthoracic Doppler echocardiographic studies will be performed in the rats every week following implantation. The rats are anesthetized as described above, the chest wall shaved, and echocardiography performed using our echo system equipped with a 7.5 MHZ transducer (Hewlett Packard Sonos 2500). A 2-dimensional short axis view of the left ventricle is obtained at the level of the papillary muscle to record M-mode tracing. Anterior and posterior end-diastolic and end-systolic wall thickness and LV diameters are measured using the American Society of Echocardiography Lineage Method, from at least three consecutive cardiac cycles. The changes in wall thickness and ventricular segmental wall motion and diameter will be recorded on videotape, and assessed by blinded independent echocardiographers.

Morphological and Immunohistochemical Studies

The heart specimens obtained from the recipient rats at various intervals will be perfused with 100 ml of saline through the posterior wall of the left ventricle, avoiding the transplant area, then processed for frozen sections. The lateral wall of the left ventricle is isolated from the remainder of the heart. Sections 6 μm thick are cut from the hearts and successive sections collected by gelatine coated glass slides. This ensures that different stains could be applied on successive sections of the tissue cut through the transplanted area (FIGS. 1 and 3). One of the sections is mounted and stained with X-Gal, to identify and view the β-gal labeled donor cells. An adjacent section is stained with hematoxylin and eosin as described in the manufacturer's specification (Sigma Diagnostics) to depict nuclei, cytoplasm and connective tissue. Other adjacent sections will be immunolabeled using various antibodies for immunohistochemical evaluation in order to identify phenotypic expression at the molecular level. These antibodies include those against myosin-slow molecules, cardiac gap junctional protein Connexin 43, desmin, and sarcomeric myosin (MF 20). Finally, specimens will also be processed and sent for ultrastructural examination in our future studies.

### EXAMPLE II

Bone marrow stromal cells (MSCs) were infused into ascending aorta of isogenic recipient rats after coronary artery ligation. MSCs were found to traffic through the coronary circulation to the injured heart, and form cardiomyocytes, fibroblasts and endothelial cells etc., depending on the specific microenvironment. Thus, autologous MSCs appear to participate in the post-infarct repair and remodeling process, and provide a therapeutically utility in treating heart failure and in improving cardiac function.

Methods

Animals

Male inbred Lewis rats 200 to 250 gm were obtained from Charles River Laboratories. These isogenic rats were used as donors and recipients to simulate the autologous infusion of MSCs in the future clinical application. All animals received humane care in compliance with the “Guide for the Care and Use of Laboratory Animals” prepared by the Institute of Laboratory Animal Resources, National Research Council, and published by the National Academy Press, revised 1996 and the “Guide to the Care and Use of Experimental Animals” of the Canadian Council on Animal Care.

Myocardial Infarction Model

12 recipient rats were anesthetized with isoflurane (MTC Pharmaceuticals). Rats were intubated and ventilated at 85 breaths/min. The heart was exposed via left thoracotomy incision. The left coronary artery was identified and ligated proximally using a 7-0 polypropylene suture. Regional myocardial ischemia was confirmed by the rapid occurrence of akinesia in the area at risk. The wound was then closed.

Isolation and Culture of Marrow-derived Stromal Cells

Isolation and primary culture of MSCs from the femoral and tibial bones of donor rats were performed according to Caplan's method (Wakitani S, Saito T, Caplan A. I. Myogenic cells derived from rat bone marrow mesenchymal stem cells exposed to 5-azacytidine. Muscle & Nerve 1995;18:1417-1426). After overdose with pentobarbital (100 mg/Kg given intraperitoneally) the femoral and tibial bones were collected. Both ends of the bones were cut away from the diaphyses. The bone marrow plugs were hydrostatically expelled from the bones with complete medium. The marrow plugs were disaggregated and the dispersed cells were centrifuged and resuspended twice in complete medium. These cells in 10 mL of complete medium were then introduced into tissue culture dishes. Medium was completely replaced every 3 days and the non-adherent cells discarded. Each primary culture was replated twice (first and second passages) to three new plates when the cell density within colonies became 80% to 90% confluent. After the twice-passaged cells became nearly confluent, they were harvested and used for the coronary infusion experiments.

Medium

The cells were routinely cultured in complete medium consisting of Dulbecco's modified Eagle's medium (DMEM) containing selected lots of 10% fetal calf serum and antibiotics (100 U/mL penicillin G, 100 μg/mL streptomycin and 0.25 μg/mL amphotericin B; all obtained from Gibro laboratories) at 37° C. in a humidified atmosphere of 5% C02.

Marrow-derived Stromal Cell Labeling

P+E86 murine ectropic retrovirus-packaging cells, which are derived from NIH 3T3 mouse fibroblasts, were obtained from Dr. Denis Cournoyer (McGill University, Montreal, QC, Canada) (Momparler R L, Laliberte J, Eliopoulos N, Beausejour C, Cournoyer D. Transfection of murine fibroblast cells with human cytidine deaminase cDNA confers resistance to cytosine arabinoside. Anti-Cancer Drugs 1996;7:266-274). The GP+E86 cells were transfected with the purified plasmid DNA pMFG-LacZ in a 10:1 molar ratio using the standard calcium phosphate transfection kit (Pharmacia, Baie d'Urfe, Quebec, Canada) . The LacZ gene encodes for the production of bacterial β-galactosidase. These cells were plated at 25% confluence for 48 hours. The second passaged MSCs growth medium was replaced with the supernatant from the GP+E86 cells (containing the replication-defective retrovirus carrying the β-gal reporter gene) to transfect the MSCs overnight and then replaced with normal complete medium for the following day. After three times of transfection, MSCs were then collected (approximately 2×106 cells for one infusion) and resuspended in 50 μL of serum-free DMEM and stored on ice until infusion into the ascending aorta. Some culture plates were selected for histochemical staining in vitro for β-galactosidase activity. The cells were fixed in 2% formaldehyde and 0.2% glutaraldehyde in phosphate buffered saline (PBS) in 4° C. for 5 minutes. Staining for β-gal was accomplished at 37° C. for 16 hr in a solution containing 1 mg/ml 5-bromo-4-chloro-3-indoyl-β-D-galactoside (X-gal), 2% dimethylsulfoxide, 10 mM potassium ferricyanide, 10 mM potassium ferrocyanide, 1 mM magnesium chloride, and 0.02% Nonidet P-40 in PBS, pH7.3.

Infusion of Marrow-derived Stromal Cells

Two weeks after the coronary ligation, the 12 recipient rats were prepared for infusion of MSCs. Anesthesia was induced and maintained as above. The rats were intubated and connected to the ventilator. The ascending aorta was exposed through upper median sternotomy and looped after dissection. Under direct vision, transfected MSCs suspension was then infused into the briefly distally clamped ascending aorta (about 20 seconds). After the infusion, the puncture bleeding site over ascending aorta was controlled by compression with gauze. The wound was then closed in layers.

Histology and Histochemical Staining for β-galactosidase Activity

Two rats were sacrificed immediately after the infusion. The hearts were excised and sliced along short axis of left ventricle to 3 mm thick sections in series, and fixed in 2% paraformaldehyde in PBS for 2 hours. The sections were then cryoembedded after protection with 20% sucrose in PBS overnight. The other 10 rats were taken for their final experiments 4 weeks after the MSCs infusion. After overdose with pentobarbital, the hearts were exposed and injected with 100 ml saline (0.9%) through the apex of the left ventricle, then perfusionfixed with 2% paraformaldehyde in phosphate buffered saline (PBS). The hearts were excised, sliced and prepared as mentioned above. Cryosections 6 μm in thickness were collected in each 3 mm section sample across a set of gelatin-coated glass slides. One of every 10 cryosections was collected for histochemical staining for β-galactosidase activity as mentioned above. The sections were then counter-stained with hematoxylin and eosin. Tissue sections were examined with an Olympus microscope (BX-FLA, Olympus). Digital images, transferred to a computer equipped with Image Pro software (Media Cybernetics, MA), were subsequently printed.

Histochemical Stain for Gap-junction Protein, Connexin 43

The gap junctions which constitute cardiac muscle specific intercalated discs were demonstrated by histochemical stain for connexin 43, using rabbit anti-connexin antibodies (Zymed Laboratories Inc., San Francisco). Diaminobenzidine was used as a chromogen to produce the brown colour which represent gap junctions linking cardiomyocytes together (Nagy J I, Li W E, Roy C, Doble B W, Gilchrist J S, Kardami E, Hertzberg E L. Selective monoclonal antibody recognition and cellular localization of an unphosphorylated form of connexin 43. Exp. Cell Res. 1997; 236:127-136).

Results

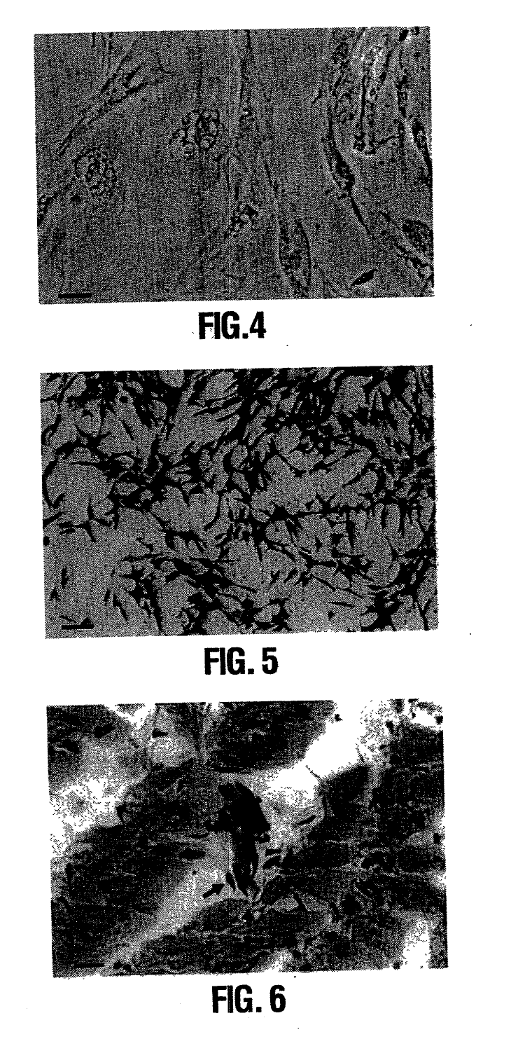

Cultured MSCs were observed by phase microscope to assess the level of expansion and to verify the morphology at each culture medium change. Most of the hematopoietic stem cells were not adherent to the culture plate and removed with changes in medium. The adherent cells were seen as individual cells or colonies of only a few cells on day 6. However, they replicate rapidly and form colonies of up to 100 cells after the first week of culture. By the end of second week, the colonies of adherent cells have expanded in size, with each colony containing several hundred to several thousand cells. Adherent MSCs from rat legs have similar morphology, most being fibroblastic in appearance, with a few adipocytic, polygonal cells (FIG. 4). This phenotype was retained throughout repeated passages under nonstimulating condition.

We transfected twice-passaged MSCs with replication-defective retrovirus carrying the β-gal reporter gene as cell labeling before their implantation. The transfected MSCs showed clear histochemical staining for β-galactosidase activity (FIG. 5). Transfection efficiency of the MSCs culture was almost 100%.

The rats were sacrificed at the following intervals after the MSCs infusion: immediately for 2 rats; and 4 weeks for 10 rats. Gross examination of the excised hearts shows clear myocardial scar formation (about 40% of LV free wall) on all 12 recipient rats. Cryosections of the specimens were selected (as detailed in Methods) for histochemical staining of β-galactosidase activity to trace and evaluate the morphology and phenotype changes of infused MSCs. Labeled cells could be identified in both rats sacrificed immediately after MSCs infusion, and in 6 out of 10 rats sacrificed 4 weeks later.

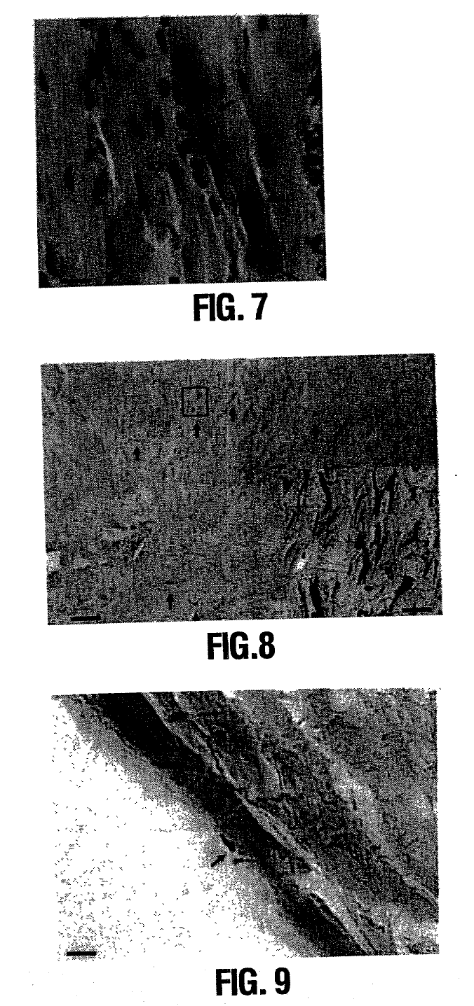

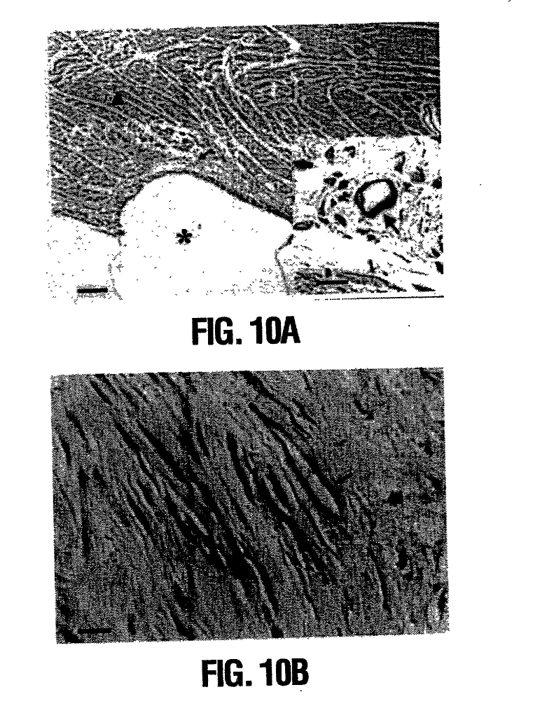

Immediately after MSCs infusion, β-gal positive cells were consistently found in all selected sections to be trapped within the coronary capillaries surrounded by endothelial cells all over the non-infarct area (FIG. 6). We failed to identify any β-gal positive cells in the infarction zone at this time. Four weeks after MSCs infusion, some β-gal positive cells could be found within the normal myocardial area outside the infarction scar (FIG. 7). They have centrally located nuclei and are connected among themselves and with surrounding host cardiomyocytes (β-gal negative cells) by intercalated disk-like structure, which are characteristics of normal cardiomyocytes. However, the β-gal positive cells also could be detected individually or in clusters within the myocardial scar (FIG. 8). They appear unorganized and scattered in the infarction scar with fibroblast-like morphology, similar to that of the surrounding β-gal negative (host) fibroblast cells. Some β-gal positive cells were found incorporated into endocardium (FIG. 9) and coronary capillary endothelium within or outside the infarct scar area (FIGS. 10A & 10B).

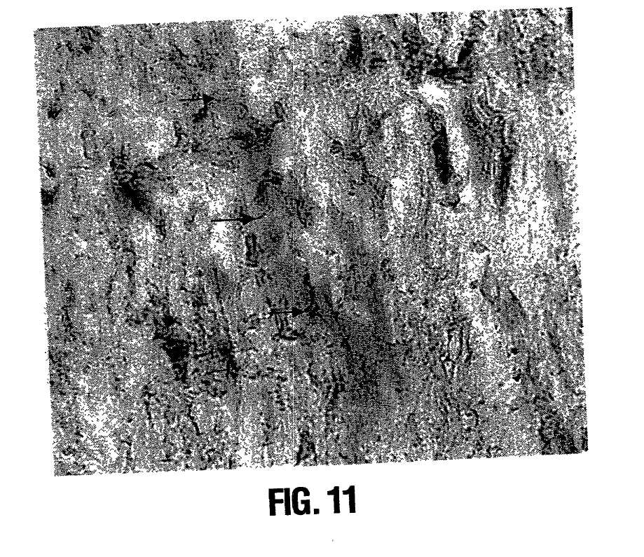

Tissue sections showing labeled cells with histological features of cardiomyocytes (as in FIG. 7) were further studied immunohistochemically using antibodies against connexin 43, a major constituent protein of gap junctions in the intercalated discs of cardiac myofibers. The demonstration of such junctional structure (FIG. 11) further confirms the phenotype of the differentiated labeled cells, and their integration into the native cardiac myofibers.

Our research on implanting mesenchymal stem cells into the myocardium indicated that these cells could be implanted into the heart by direct injection, either from the surface of the heart (epicardium) or from inside the ventricle (endocardial). However, we also found that the cells can be injected into the coronary arteries directly which could then translocate outside of the vascular system, and differentiate into cardiac myocytes.

Accordingly, coronary artery delivery bringing the cells throughout the heart muscle is provided in connection one aspect of the present invention. Furthermore, the present invention also provides a method of delivering MSCs to other damaged organs or tissues in the body when injected intravascularly (as discussed further below).

This technique is particularly desirable in treating patients with diffuse cardiomyopathy. Diffuse cardiomyopathy describes heart diseases caused by various etiologies, such as viral infection, drug toxicity (such as by chemotherapeutic agent adriamycin), endocrine, metabolic or hereditary diseases, with the common denominator that all of these causes result in diffuse injury of the heart muscle. In contrast to heart muscle damage caused by myocardial infarction secondary to coronary artery occlusion, there is no localized scar formation. Since all the muscles in the heart are involved, to distribute the mesenchymal cells diffusely throughout the heart muscle via the coronary artery is ideal. The clinical importance of being able to treat diffuse cardiomyopathy is due to the fact that 1) there is no effective surgical procedure except heart transplantation which can surgically treat patients with heart failure caused by diffuse cardiomyopathy, once drug therapy fails. This is in contrast to myocardial infarction, in which many of the patients can be treated with various surgical therapies, such as coronary artery bypass surgery, and possibly transmyocardial revascularization. The statistics show that approximately half or more of the patients who undergo heart transplantation suffer from diffuse cardiomyopathy and not coronary artery disease. 2) The marrow stromal cells implanted into the heart, either by direct injection or through the coronary, received the signals to guide their differentiation from their microenvironment, namely, the tissue immediately surrounding them. Thus, when the implanted cells are in close proximity to the native cardiac myocytes, they differentiate into heart muscle cells themselves, but when they are implanted into the middle of the scar surrounded by fibrous tissue, they could differentiate into connective tissue like their surroundings. 3) We have devised a strategy to deal with this situation. Instead of injecting the mesenchymal stem cells obtained and cultured in vitro directly into the heart, these cells may be chemically modified during culture prior to implantation in order to guide them to differentiate into cardiac myocytes such that they no longer respond to the micro-environment, and continue their differentiation to become heart muscle cells, even within scar tissue. These marrow stem cells are isolated and cultured overnight, and then treated with 5 mmol/lt of 5-azacytidine (Sigma Company), or 1 mmol/lt of 5-aza-2′deoxycytidine and incubated for 24 hours. These demethylating agents have been shown to be able to convert marrow stromal cells to express cardiomyocyte phenotype in vitro. These cells are then used for implantation into the scar zone.

One strategy of implantation for patients who suffer from myocardial infarction and heart failure is to use such modified marrow stromal cells for direct multiple injection into the scar zone, supplemented by injection or delivery of non-modified marrow stromal cells to the non-infarcted myocardium. The rationale for doing so is twofold: 1) when there is large scar tissue, the remaining myocardium tries to compensate for the loss of function by the scar tissue, and undergoes a remodeling process of dilatation. Adding new myocytes into the myocardium surrounding the scar tissue can reduce the stress on each individual myocyte as the result of ventricular dilatation. Secondly, with the need for collateral blood flow, we have observed that these unmodified marrow stromal cells can also differentiate into endothelial tubes and smooth muscle fibers which are part of the angiogenesis to provide the necessary blood flow for implanted cells within and around the scar tissue. Clinically, it is possible to accomplish these implantations during a single cardiac catheterization session. Using a routine coronary angiograph catheter, the marrow stromal cells can be delivered into the coronary circulation which would go primarily to non-scar tissue because the coronary artery to the infarcted segment of the heart has already occluded. Then, modified marrow stromal cells can be injected endocardially into the infarct segment under fluoroscopic and echocardiographic guidance, via, for example, an appropriately designed endocardial implantation catheter. In order to accomplish this, the bone marrow stromal cells harvested from the patient prior to implantation would be cultured, multiplied and separated into two groups. One set of the marrow stromal cells will be cultured and multiplied without modification; while the other culture plates will receive 5-azacytidine or 5-aza-2′-deoxycytidine to initiate their differentiation process toward cardiac myocytes in vitro, as described above. These cells will be harvested separately and injected either into the coronary system or the myocardial scar zone respectively.

According to one embodiment of the present invention, a method of treating myocardial infarction/heart failure is provided. The superiority of this method is it effectively accomplishes three goals at the same time, namely, 1) to repopulate the scar tissue with cardiac myocytes, 2) to augment the non-infarcted myocardium to modulate the remodeling process, and 3) to enhance angiogenesis to promote the development of collateral circulation.

### EXAMPLE III

### Transvenous Bone Marrow Stromal Cell Transplantation for Therapy of Multiple Organ Failures

Preliminary Studies

To test our hypothesis that a myocardial infarction with the death of a portion of the heart muscle could send a signal to mobilize marrow stromal cells, migrating to the injured heart where they could differentiate to ameliorate the damage incurred, we did two series of experiments.

Materials and Methods

Isogenic adult rates were used as donors and recipients to stimulate the autologous repair process after myocardial infarction (MI). MI was created by proximal occlusion of left coronary artery in 12 recipient rats. MDSCs isolated from donor leg bones were purified, expanded, and retrovirally transfected with Lac Z reported gene for cell labeling. Such labeled MDSCs were then injected into the briefly distally clamped ascending aorta of recipient rats 2 weeks after their left coronary artery had been ligated. The hearts were harvested immediately (n=2) or 4 weeks (n=10) after the cell injection for studies to trace the implanted cells and identify their phenotypes.

Results

Our results indicate that these cells can be delivered by coronary blood flow to various parts of the heart, including the infarcted scar tissue zone, the non-infarcted healthy myocardial segment, as well as the border zone between these two segments. We found that the labeled cells differentiated according to the cues from the micro-environment, and matured into cardiomyocytes, endothelial cells in capillaries, and some scar tissue. This indicates that the marrow stromal cells are capable of contributing to all three pathophysiological processes which take place following myocardial infarction, namely neoangiogenesis to develop collateral circulation to the infarcted ischemic zone, remodeling of the non-infarcted myocardium to compensate for the lost muscle mass, and perhaps to reduce the scar expansion and rupture.

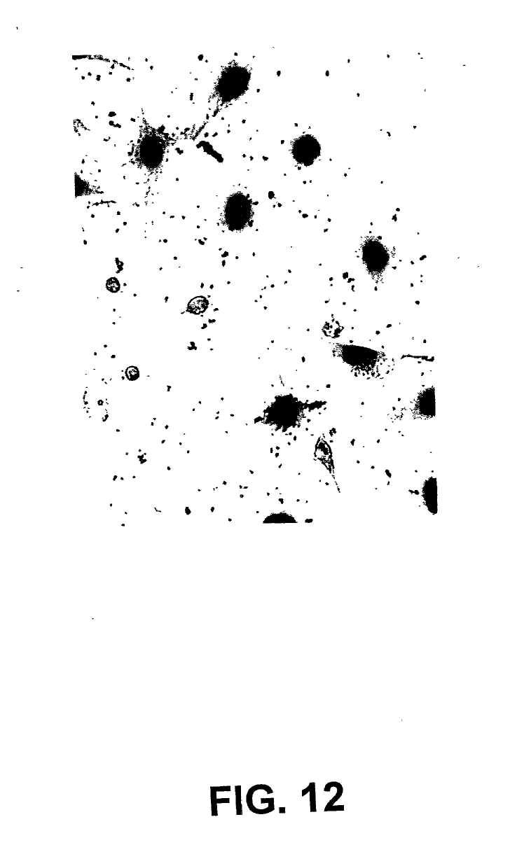

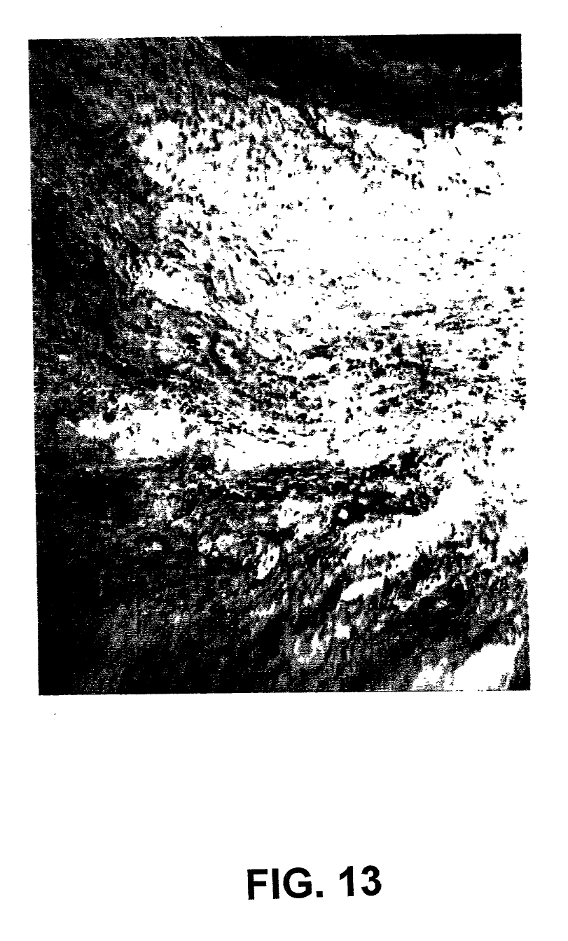

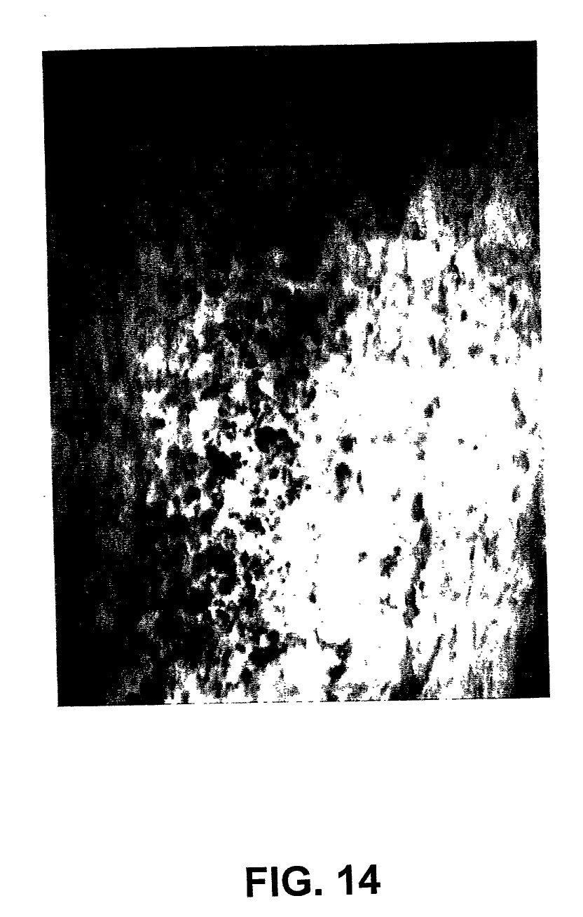

A second series of experiments were conducted to determine whether, in a spontaneous myocardial infarction as seen clinically, marrow stromal cells can indeed be mobilized to migrate to the injured heart via the circulatory system. In this series of experiments, marrow stromal cells were isolated, culture expanded and labeled before they were harvested for injection intravenously to an isogenic rat. As shown in FIG. 12, the bone marrow taken from the recipient rat 24 hours later showed that these cells had homed into the bone marrow, since we were able to identify numerous labeled cells in the bone marrow at this time. When the hearts were removed from such animals one to six weeks after transvenous marrow stromal cell transplantation, no labeled cells could be found in these normal heart tissues. However, if we ligated the coronary artery to produce myocardial infarction in animals which already have labeled marrow stromal cells in the bone marrow, we were able to identify these labeled cells appearing in and near the infarcted myocardium, suggesting that they had been recruited and migrated from the bone marrow to the damaged organ. The cell labeling technique used in this experiment was transfection with retrovirus carrying the lac-G gene which produces β-galactosidase in the labeled cell. When such cells are stained with X-gal, the blue color indicates the presence of β-galactosidase activities. FIGS. 13 and 14 show numerous labeled cells at the scar and border zone of the myocardial infarction. This specimen was also stained immunohistochemically against troponin 1-C which is specific to cardiac myocytes. The heavily brownish stained cells in the periphery of the picture are surrounding normal heart muscles, while the central scar area appears to also show brown color in the cytoplasm of labeled cells, indicating the synthesis of cardio-specific troponin 1-C.

Conclusions

In accordance with these findings, the following conclusions are provided: 1) The myocardial infarction sends a signal to the bone marrow (such molecular mechanisms may be cytokines or growth factors, for example). 2) The marrow stromal cells are mobilized, and reach the damaged heart via the circulatory system (i.e. coronary arteries). 3) The marrow stromal cells are targeted toward the injured myocardium and the neighboring border zones, and undergo differentiation, expressing their phenotypes.

It is fully contemplated that these findings with respect to MSCs may be applied in the treatment of many types of organ and tissue failure, such as, for example the liver or brain, whereby MSCs are recruited to various sites in vivo and undergo site specific differentiation to repair damaged tissues/organs etc. Furthermore, MSC may be recruited to enhance angiogenesis in ischemic skeletal muscle.

We have shown that marrow stromal cells can be infused intravenously and they will home into and reside in the bone marrow where they serve as a reserve for organ repair. From there they can be recruited to various organs and undergo site specific differentiation to repair damaged tissues. Accordingly, in patients who suffer or are likely to suffer multiple organ failures, we propose to obtain autologous marrow stromal cells, culture and vastly expand the population of the cells in vitro, in order to infuse them back to the patient, augmenting the repair capability for damaged organs. Thus, in accordance with the present invention there is provided a multi-purposes therapy for repairing tissue and organ damage in vivo and preventing or ameliorate multiple organ failure and death. Furthermore, it may also be possible to use allogeneic or xenogeneic marrow stromal cells as donors for intravenous therapy as described in this invention.

### EXAMPLE IV

### Organ Repair Using Allo-/Xeno-Transplantation of Bone Marrow Stromal Cells Without Immunosuppression

Preliminary Studies

A technique of implanting autologous bone marrow stromal cells into the myocardium to grow new heart muscle cells was studied, as discussed above (Wang et al. Journal of Thoracic and Cardiovascular Surgery. November 2000, Vol. 120, No. 5, 999-1006). These experiments were carried out in rats. In order to prepare doing similar experiments in larger animals, namely pigs, prior to our clinical trial, we harvested bone marrow cells from a piglet using a marrow aspiration technique, and isolated the stromal cells using Caplan's technique, as described in Wang et al. The original goal was simply to test the feasibility of isolating, culturing and expanding pig stromal cells to see if our technique used for these procedures in rats could also be successfully applied to pigs. Indeed, these porcine cells grew and proliferated in culture, thus confirming that the procedures and methodology as discussed above had successful application in other species. We proceeded to label these cells in culture, and implanted them by direct needle injection into the myocardium of a rat, in accordance with our previous rat-to-rat transplant protocol. The cell labeling was accomplished by adding an agent “DAPI”, which binds with the DNA in the cell nucleus to become fluorescent in order to identify and trace the donor cells after their implantation into the host myocardium.

This methodology was repeated in a similar experiment where bone marrow stromal cells (MSCs) obtained from pigs were cultured, expanded, labeled with DAPI or beta-Gal, and then injected intravenously into the rat penile veins.

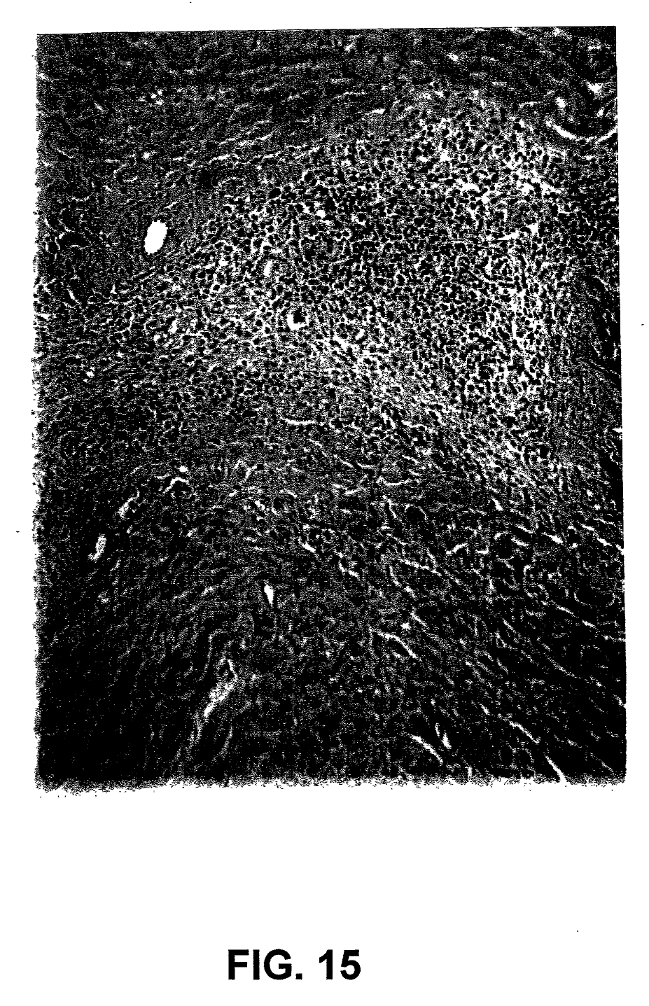

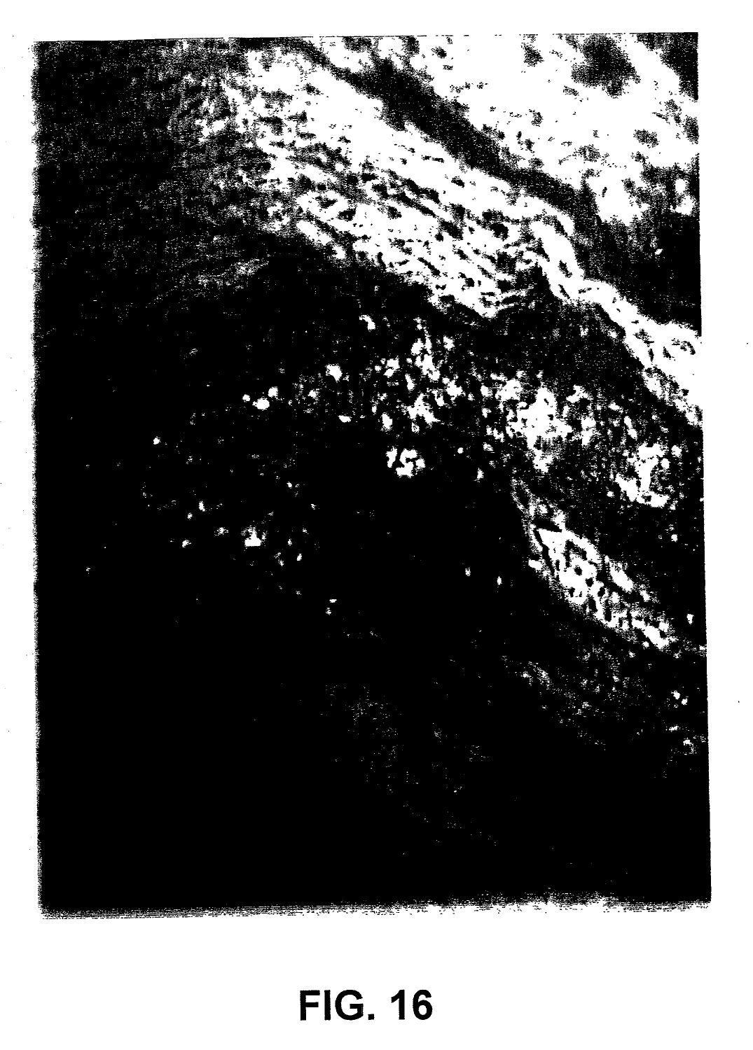

Within days, the labeled MSCs were found in the rat's own bone marrow as illustrated in FIG. 15. In control rats, the undamaged hearts contained no labeled cells. In the experimental group of rats, the anterior coronary artery was ligated to create a myocardial infarction. Within days, labeled cells, presumably recruited from the bone marrow, were found in and near the infarct site of the myocardium, as shown in FIG. 16. These cells may undergo in situ, milieu-dependent differentiation to express various phenotypes, and participate in the healing process of the heart, by strengthening the scar tissue to prevent infarct rupture, by participating in angiogenesis to enhance collateral blood flow, and by augmenting the non-infarcted myocardium to modulate the ventricular remodeling process. There are evidence that similar phenomena will happen when organs other than heart are damaged.

Results

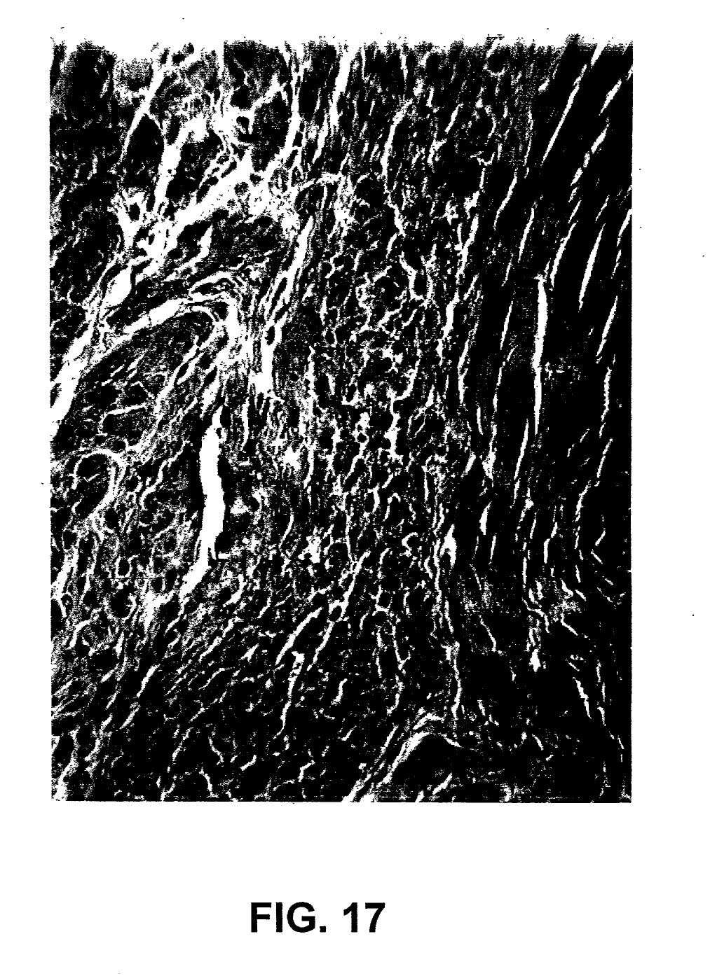

In effect, what we did was to transplant pig stem cells into rat heart muscle, either directly or intravenously, as described above. According to the existing knowledge, such xeno-transplantation should be rejected by the immunologically competent normal adult rats. To our surprise, these pig stem cells survived, and appeared to differentiate, expressing certain proteins specific to heart muscle cells. In the attached photograph FIG. 15 illustrates a histological section of the site of the rat heart where pig stromal cells were implanted two weeks prior. The photograph illustrated in FIG. 15 was taken following hematoxylin and eosin stain, showing numerous cells at the injection site surrounded by rat myocardium. An adjacent histological section which was observed under fluorescent microscopy is shown in FIG. 16. Faint background fluorescence of the cardiac muscles can be seen surrounding the implant site. There are clearly fluorescent cells present at the implant site, indicating they are cells derived from pig stromal cells. If these cells did not survive, they would have been removed by phagocytes at the two week post implantation point. In additional specimens the labeled donor cells are still visible at four to six weeks after implantation, indicating that they in fact survived. FIG. 17 depicts an immunohistochemical staining of such a specimen at four to six weeks post implantation, using antibodies against troponin 1-C. Troponin 1-C is specific to the heart muscle, and is part of the molecular structure of the contractile apparatus of a heart muscle cell. In the peripheral area of FIG. 17, longitudinal cardiac muscle fibers staining positive (brown color) for troponin 1-C can be seen, as expected. Of interest is the cells at the implant site, which also show a brown stain in their cytoplasms, indicating that these cells were able to synthesize troponin 1-C, which is specific to normal heart muscle, suggesting that they are differentiating and expressing some characteristics of a cardiac myocyte.

Similar results were obtained, without evidence of rejection when the above methodology was used in a transplant of murine marrow stromal cells into rat. The murine MSCs were shown to survive in the rat bone marrow for more than two weeks, without rejection.

It is fully contemplated that other techniques for identifying these cells in viva, such as other cell labeling techniques for example, may be employed in connection with the present invention.

Conclusions

In view of these results, as discussed above, it is concluded in accordance with the present invention that marrow stromal cells can survive following their transplantation into another species, or another subject of the same species and undergo site specific differentiation as observed in autologous MSC transplants.

As mentioned earlier, normally donor cells from a different species are rapidly rejected by the host immune system as they recognize specific antigens expressed on the donor cell surfaces. This is also true with a large number of donor cells from other subjects of the same species. Our observation indicates that this rule does not apply when the donor cells are primitive, undifferentiated stem cells. We propose two possible explanations. The first is that the donor cells interact with the immune system of the recipient to make the latter incapable of reacting to and rejecting the donor cells. Given that we implanted xeno-geneic cells in a limited number within a small area of the heart, it is difficult to imagine that they can affect the immune system as a whole in the host animal. However, local interaction between the donor cells and host cellular infiltrates cannot be ruled out. An alternative mechanism is that the cells expressed their identifying specific cell surface antigens as they differentiate from stem cells into mature cells. It is postulated, however, that such antigen expression could be modulated by the micro-environment of these differentiating stem cells. We, therefore, conclude that such non-embryonic stem cells derived from bone marrow can be transplanted into another species, or another subject of the same species and without immunosuppression of the host, they can develop trans-species tolerance to survive and differentiate. Since marrow stromal cells have been shown to be able to undergo site specific differentiation in multiple organs ranging from the liver, bone, muscle, lungs, and even the brain, we propose the use of allo- or xeno-transplantation of bone marrow stromal cells without immunosuppression for eliciting tissue cell growth and organ repair in vivo, in accordance with the present invention.

Our observations, indicate that allo- and xeno-tranplantation of MSCs could be carried out in immunologically competent, mature host subjects to achieve in viva tissue cell growth and/or organ repair without requiring immunosuppression.

While the invention has been described in connection with specific embodiment thereof, it will be understood that it is capable of further modifications and this application is intended to cover any variations, uses or adaptations of the invention following in general, the principles of the invention and including such departures from the present disclosure as come within the known customary practice within the art to which the invention pertains and as may be applied to the essential features hereinbefore set forth, and as follows in the scope of the appended claims.

What is claimed is:

1. A method of inducing tissue cell growth and/or organ repair in vivo without eliciting an immune response; which comprises the step of transplanting undifferentiated stem cells from a host of one species into a recipient of the same species.

2. The method of claim 1, wherein the undifferentiated stem cells are bone marrow stroma cells.

3. The method of claim 1, wherein said undifferentiated stem cells are systemically transplanted into said same species.

4. The method of claim 1, wherein said undifferentiated stem cells are locally transplanted into a site of tissue cell or organ damage in said same species.

5. The method of claim 3, wherein said undifferentiated stem cells are transplanted by transvenous infusion.

6. The method of claim 4, wherein said undifferentiated stem cells are transplanted by in vivo topical administration.

7. The method of claim 1, wherein when said undifferentiated stem cells are transplanted in vivo, and the undifferentiated stem cells are differentiated into a cell type of a damaged tissue and/or organ.

8. The method of claim 7, wherein said cell type includes cardiac myocytes, skeletal myocytes, nerve cells, liver cells and bone cells.

9. The method of claim 1, which further comprises the step of using a cell labeling technique to confirm survival and differentiation of implanted stem cells, and to identify a phenotype of said stem cells by both morphology and molecular markers.

10. The method of claim 1, which further comprises examining the effects of the micro-environment of implanted stem cells on their differentiation and phenotype expression.

11. A method of inducing tissue cell growth and/or organ repair in vivo without eliciting an immune response; which comprises the step of xeno-transplanting undifferentiated stem cells from a first species into a second species.

12. The method of claim 11 wherein said undifferentiated stem cells are bone marrow stroma cells (MSCs).

13. The method of claim 11 wherein said undifferentiated stem cells are systemically transplanted into said second species.

14. The method of claim 11 wherein said undifferentiated stem cells are locally transplanted into a site of tissue cell or organ damage in said second species.

15. The method of claim 13, wherein said undifferentiated stem cells are transplanted by transvenous infusion.

16. The method of claim 14, wherein said undifferentiated stem cells are transplanted by in vivo topical administration.

17. The method of claim 11, wherein when said undifferentiated stem cells are transplanted in vivo, the undifferentiated stem cells are differentiated into a cell type of a damaged tissue and/or organ.

18. The method of claim 17, wherein said cell type includes cardiac myocytes, skeletal myocytes, nerve cells, liver cells and bone cells.

19. The method of claim 11, which further comprises the step of using a cell labeling technique to confirm survival and differentiation of implanted undifferentiated stem cells, and to identify said undifferentiated stem cells phenotype by both morphology and molecular markers.

20. The method of claim 19, which further comprises examining the effects of the micro-environment of implanted undifferentiated stem cells on their differentiation and phenotype expression.

21. A method or treating tissue and/or organ damage in vivo, said method comprising:
(a) retrieving bone marrow from a host;
(b) isolating marrow stroma cells from said bone marrow of said host;
(c) expanding said marrow stroma cells in culture; and
(d) transplanting said marrow stroma cells into a recipient suffering from tissue and/or organ damage.

22. The method of claim 21 wherein said host is selected form a species of the recipient or another species.

23. The method of claim 21 wherein said step of retrieving bone marrow includes performing a bone marrow puncture.

24. The method of claim 21 wherein said step of transplanting said marrow stroma cells the recipient includes transplanting said cells into a site of tissue and/or organ damage.

25. The method of claim 24 wherein said step of transplanting includes selective infusion.

26. The method of claim 24 wherein said step of transplanting includes tranevenous catheter injection.

27. The method of claim 24 wherein said site of tissue and/or organ damage is the myocardium.

28. A method of treating cardiac failure in a patient, said method comprising:
(a) retrieving bone marrow from one of a host or said patient suffering from cardiac failure;
(b) isolating morrow stroma cells from said bone marrow;
(c) expanding said marrow stroma cells in culture;
(d) treating said marrow stroma cells in culture to inducea cell phenotype capable of enhancing cardiac function; and
(e) transplanting said marrow stroma cells into a myocardium of said patient suffering from cardiac failure.

29. The method of claim 28 wherein said host is selected from one of a species of said patient or another species.

30. The method of claim 28 wherein said step of treating said marrow stroma cells includes treating said cells with at least one of 5-azacytidine and 5-aza-2′-deoxycytidine.

31. The method of claim 28 comprising further treating said myocardium with non-modified marrow stroma cells.

32. The method of claim 28 wherein said cell phenotype is a cardiomyocyte.

33. The method of claim 28 wherein said cell phenotype is capable of enhancing angiogenesis.
